(19) United States
(12) Patent Application Publication
(10) Pub. No.: US 2014/0314846 A1
(43) Pub. Date: Oct. 23, 2014

(54) MULTIPLE UNIT PHARMACEUTICAL FORMULATION

(71) Applicants: Adel PENHASI, Holon (IL); Avi AVRAMOFF, Haifa (IL); Mila GOMBERG, Jerusalem (IL); Sheera MOSES-HELLER, Atlit (IL)

(72) Inventors: Adel PENHASI, Holon (IL); Avi AVRAMOFF, Haifa (IL); Mila GOMBERG, Jerusalem (IL); Sheera MOSES-HELLER, Atlit (IL)

(73) Assignee: Dexcel Pharma Technologies Ltd., Or- Akiva (IL)

(21) Appl. No.: 14/168,911

(22) Filed: Jan. 30, 2014

Related U.S. Application Data
(63) Continuation of application No. 12/227,864, filed on Jul. 21, 2010, now abandoned.
(60) Provisional application No. 60/809,942, filed on Jun. 1, 2006.

(51) Int. Cl. A61K 9/28; A61K 31/4439

(52) U.S. Cl. CPC A61K 9/2886; A61K 31/4439
USPC 424/465; 514/338

(57) ABSTRACT

An orally disintegratable benzimidazole formulation, featuring a plurality of compressed pellets in a MUPS tablet. The individual units feature a substrate with the active ingredient and an enteric coating, optionally with a subcoating between the substrate and the enteric coating. The individual units are preferably at least partially coated with an outer coating which features a stress absorber, thereby enabling the pellets to be compressed without disturbing the integrity of the enteric coating. The enteric coating preferably does not feature a plasticizer.

### FIELD OF THE INVENTION

The present invention relates to a novel formulation for a benzimidazole, and methods of preparation and administration thereof, and in particular, to an individually enteric-coated multiple unit formulation.

### BACKGROUND OF THE INVENTION

Omeprazole, Pantoprazole, Lansoprazole and other derivatives of benzimidazole, which are active proton pump inhibitors and used conventionally for decreasing gastric secretion, are known to be susceptible to degradation and transformation in acid media.

Lansoprazole, 2-[[(3-methyl-4-(2,2,2-trifluoroethoxy)-2-pyridyl)methyl]sulfinyl]benzimidazole is described for example in U.S. Pat. Nos. 4,628,098, and 4,689,333 and European Patent No. 174726.

Another popular benzimidazole derivative, Omeprazole, 5-methoxy-2(((4-methoxy-3,5-dimethyl-2-pyridinyl)methyl)sulfinyl)-1H-benzimidazole, is disclosed and described in European Patent No. 5129 and European Patent No. 124495, as well as in numerous other patents and published patent applications.

The susceptibility of these active proton pump inhibitor substances to degradation and transformation in acid media increases the difficulty of preparing a pharmaceutical form designed for oral administration. If the active substance comes into contact with the stomach content, which is a highly acidic medium, these chemical substances become degraded. Thus, these benzimidazoles should be protected both during storage and during their passage through the acidic environment of the stomach.

European Patent No. 237200 discloses one solution, which is to directly coat the solid core containing the benzimidazole with an enteric coating layer.

Enteric coating layers are formed by use of enteric polymers, such as cellulose, vinyl, and acrylic derivatives. These polymers exhibit resistance to gastric fluids, yet are readily soluble or permeable in intestinal fluid. Enteric polymeric materials are primarily weak acids containing acidic functional groups, which are capable of ionization at elevated pH. In the low pH of the stomach, the enteric polymers are unionized, and therefore, insoluble. As the pH increases in the intestinal tract, these functional groups ionize, and the polymer becomes soluble in the intestinal fluids. Thus, an enteric polymeric film coating allows the coated solid to pass intact through the stomach to the small intestine, where the drug is then released for absorption through the intestinal mucosa into the human body where it can exert its pharmacologic effects.

However, this apparent solution to the instability of benzimidazoles caused further complications, in that the alkaline core containing the benzimidazole was found to react with the enteric coating, thereby causing the enteric coating to degrade. A solution to these further complications is disclosed in United Kingdom Patent Application No. 2,189,698, in which the benzimidazole is contained within a solid active core, which is coated first with a subcoating layer and then with an enteric coating layer. The enteric coating layer protects the benzimidazole during the passage through the stomach, while the sub-coating layer protects the enteric coating layer from reacting negatively with the alkaline core containing the benzimidazole.

Oral dosage forms can be classified into two types: single unit and multiple unit. The above documents teach single unit forms comprising a single core, which may comprise a neutral core coated with a layer containing the active ingredient, or an active core in which the active ingredient is admixed with the core excipients.

Multiple-unit dosage forms have been accepted to provide advantages over single unit dosage forms. The multiple-unit dosage forms consist of many small particles, which are contained in a capsule or a tablet. The small particles are mixed with the contents in gastrointestinal tract and are distributed over a large area. Thus, high-local concentration of the drug is avoided, and the risk of local irritations is reduced.

Multiple unit dosage forms are essential where drug excipients or drug-drug physicochemical interaction is possible in a single-unit formulation; they are also known to have less variance in transit time through the gastrointestinal tract than single-unit dosage forms. Multiple-unit forms offer more predictable gastric emptying, which is less dependent on the state of nutrition, a high degree of dispersion in the digestive tract, less absorption variability, and a lesser risk of dose dumping. However, problems arise when enteric coating layered pellets containing acidic susceptible benzimidazoles as an active substance are compressed into tablets.

If the enteric coating layer does not withstand the compression of the pellets into a tablet the susceptible active substance will be destroyed by penetrating acidic gastric juice, i.e. the acid resistance of the enteric coating layer of the pellets will not be sufficient to protect the active ingredient in the tablet after compression. Such problems are typically caused by brittleness of the enteric coating, which causes cracks to form in the coating under the pressure of compression.

Plasticizers are materials having lower molecular weights than those of enteric polymers, and are commonly included in the enteric coating layer to increase separation between the polymer chains, thereby reducing the stiffness and brittleness of the coating layer, thus preventing cracking.

However, the use of a plasticizer in the coating layer is associated with a number of disadvantages. Hydrophobic plasticizers will create problems in enteric coating solution preparation due to poor solubility in aqueous solvents, and can affect the dissolution profile of the finished product. Higher concentrations of plasticizer in the coating generally tends to increase the water vapor permeability, and also to reduce the tensile strength of the coating layer. Higher concentration of plasticizer can also lead to bleeding of the plasticizer, giving an oily feel to the tablet surface. Volatile plasticizers such as propylene glycol may be largely lost due to drying during the coating process.

U.S. Pat. No. 5,464,632 teaches a rapidly disintegratable multiparticulate tablet, comprising a plurality of microcrystals or microgranules. The disintegration rate is obtained due to a mixture of excipients or vehicles which comprises at least a disintegrating agent and a swelling agent, which are mixed with the active substance. The crystals or granules may be enteric coated and formed into tablets by compression. This tablet would not be expected to be sufficiently resistant to gastric acid penetration to be suitable for use with a highly acid-sensitive benzimidazole, nor is such an acid-sensitive material taught as a suitable active ingredient.

U.S. Pat. No. 6,740,339 teaches rapidly disintegrating solid preparations. U.S. Pat. No. 4,786,505 teaches enteric-coated tablets comprising omeprazole, which do not include the use of a plasticizer in the enteric coating. Neither of these documents teaches individually enteric-coated multiple units which are compressed into a tablet.

U.S. Patent Application No. 2004/0213847 teaches an oral pharmaceutical composition in an enteric-coated solid dosage form. Multiple unit dosage forms are not disclosed. Furthermore, the enteric coating in this formulation includes a plasticizer.

U.S. Pat. No. 5,985,322 discloses an enteric formulation of the anti-depressant drug, fluoxetine. Again, multiple unit dosage forms are not taught, and the enteric coating is stated as requiring the addition of a plasticizer.

PCT Application No. WO 06/012634 teaches dosage forms with an enterically coated core tablet. Multiple unit dosage forms are not taught, and the enteric coating disclosed in the examples includes a plasticizer.

U.S. Patent Application No. 2002/0142034, PCT Application No. 99/59544 and European Patent Application No. EP 1121103 teach orally disintegratable tablets comprising fine granules, which are coated with an enteric coating layer, and may be compressed into tablets. The tablets include a plasticizer in the enteric coating layer.

U.S. Patent Application No. 2004/1031675 teaches a method of manufacturing a tablet containing coated granules by compression, such that the coating film can be prevented from rupture. Examples of suitable enteric coating layers are given as including at least one plasticizer.

U.S. Pat. No. 5,798,120 teaches tablets containing enteric-coated granules, which require the use of a plasticizer.

U.S. Patent Application No. 2006/0018964 teaches a multiparticulate tablet enteric coated particles, and a mixture of tableting excipients, comprising xylitol and/or maltitol, a disintegrating agent, a lubricant and at least one other diluent. The enteric coating includes at least one plasticizer.

U.S. Pat. No. 5,753,265 teaches a multiple unit tableted dosage form, comprising individually enteric-coated layered units, compressed into a tablet. The enteric layers are stated as containing pharmaceutically acceptable plasticizers to obtain the desired mechanical properties.

U.S. Patent Application No. 20040131675 teaches a method of manufacturing a tablet, which comprises compressing coated granules containing a physiologically active substance, at a temperature exceeding room temperature, whereby the tablet can be prevented from rupture of a part of a coating film of the granules at the time of tablet compression.

U.S. Pat. No. 7,041,316 teaches an enteric-coated pharmacological dosage form which comprises a core tablet formed by dry mixing, without using an aqueous solution. The use of a plasticizer in the enteric coating is taught. No mention is made of the use of individually enteric-coated multiple units which are compressed into a tablet. U.S. Pat. No. 5,232,706 teaches an oral pharmaceutical preparation for omeprazole, comprising a nucleus, a first coating, and a second, enteric coating. Multiple unit dosage forms are not taught.

U.S. Pat. No. 6,733,778 teaches an ompeprazole formulation comprising an active core which is directly enteric coated without the use of a separating layer between the core and the enteric coating. The formulation may further comprise a seal coat containing color, applied over the enteric coating. The use of multiple unit dosage forms is not taught, and the seal coat does not serve to protect the integrity of the enteric coating.

U.S. Pat. No. 5,817,338 teaches a multiple unit tableted dosage form of omeprazole, in which pellets or granules are covered with an enteric layer comprising a plasticizer.

U.S. Pat. No. 6,780,435 teaches an omeprazole formulation comprising a pellet and a single layer of enteric coating. A seal coating may be applied to the pellets, which does not serve to protect the integrity of the enteric coating. Compression of the pellets to form a tablet is not taught.

U.S. Pat. No. 6,576,258 teaches a pharmaceutical formulation for acid-sensitive active substances which are stabilized by anhydrous granulation. The formulation comprises pellets or granules which are compressed into tablets and subsequently coated with an enteric material. The use of particles or granules which are individually coated prior to compression is not taught.

U.S. Pat. No. 6,228,400 teaches pharmaceutical formulations for benzimidazole derivatives, comprising granules which may be compressed into tablets. The granules are individually coated with an enteric layer which includes a plasticizer. The use of an outer coating to protect the integrity of the enteric layer is not taught.

U.S. Pat. No. 6,551,621 teaches omeprazole microgranules each comprising an active layer and an outer enteric layer. The enteric layer includes a hydrophobic plasticizer. Compression of the microgranules to form a tablet is not taught; nor is the use of an outer layer to preserve the integrity of the enteric coating.

U.S. Pat. Nos. 6,136,344, 6,183,776 and 6,132,770 teach pharmaceutical dosage forms comprising an acid susceptible proton pump inhibitor in a multiple unit dosage form, which is enteric-coated. The enteric coating layer has mechanical properties such that the acid resistance of the enteric coated pellets is not significantly affected by compression of the pellets during tableting. The enteric coating comprises a plasticizer.

Drug Dev. Ind. Pharm. 24(8): 737-746 (1998) discusses the compactability of beads for oral dosage forms. Multi-layered beads, consisting of several layers of acetaminophen and polymer coating were studied, having an outer layer of mannitol as a cushioning excipient. Caplets having an outer layer of Avicel PH-101 or polyethylene oxide (PEO), and a center layer of polymer-coated beads are described as exhibiting fracturing of the polymer coating.

Drug Dev. Ind. Pharm. 25(5): 635-652 (1999) studies the prevention or mitigation of polymer coat fracture on compaction of sustained-release beads, without the addition of cushioning excipients. Swellable polymers, such as PEO, were found to prevent polymer coat rupture, but cracks did occur, which were sealed by the PEO. Polymer coatings overcoated with polyethylene glycol and microcrystalline cellulose, with an additional coating of a disintegrant, were found to partially disrupt on compaction. Ethylcellulose-coated beads granulated with cushioning excipients were also found to result in a ruptured polymer coat on compaction.

### SUMMARY OF THE INVENTION

The background art does not teach or suggest a pharmaceutical preparation for benzimidazoles, having individually enterically-coated multiple units, wherein the enteric coating is covered by an outer coating which provides resistance to cracking of the enteric coating during compression, and which is devoid of a plasticizer.

There is thus a widely recognized need for, and it would be highly advantageous to have, multiple unit enteric-coated preparations, particularly for benzimidazoles but optionally for an active ingredient with a bitter or unpleasant taste, which are devoid of at least some of the limitations that are known in the art.

The present invention overcomes these limitations by providing a novel, rapidly orally disintegratable, composition for a benzimidazole, wherein each of the individual enteric-coated multiple units is entirely coated with an outer layer which protects the integrity of the enteric coating during compression.

According to one aspect of the present invention there is provided a composition for a benzimidazole, comprising a rapidly orally disintegratable tablet having a multiplicity of compressed units, wherein each unit comprises a substrate comprising the benzimidazole; an enteric coating, which is devoid of a plasticizer, layered on the substrate; and an outer coating layered on substantially an entirety of the enteric coating.

According to another aspect of the present invention there is provided a composition for a benzimidazole, comprising a rapidly orally disintegratable tablet having a multiplicity of compressed units, wherein each unit comprises a substrate comprising the benzimidazole; an enteric coating, layered on the substrate; and an outer coating, which protects the integrity of the enteric coating, layered on substantially an entirety of the enteric coating.

According to another aspect of the present invention there is provided a composition for a benzimidazole, comprising a rapidly orally disintegratable tablet having a multiplicity of compressed units, wherein each unit comprises a substrate comprising the benzimidazole; an enteric coating, layered on the substrate; and an outer coating layered on substantially an entirety of the enteric coating, which prevents direct contact between the units and thus protects the integrity of the enteric coating.

According to another aspect of the present invention there is provided a composition for a benzimidazole, comprising a tablet having a multiplicity of compressed units, wherein each unit comprises a substrate comprising the benzimidazole; an enteric coating, layered on the substrate; and an outer coating layered on the enteric coating for binding the units during compression. The presence of the outer coating provides a solution to the problem of segregation which may occur between the units and other tabletting excipients if present in a simple mixture (as for a prior art MUPS formulation), due to differences in particle size, density, surface tension and shape.

According to another aspect of the present invention there is provided a rapidly orally disintegratable composition for a benzimidazole, comprising a tablet having a multiplicity of compressed units, wherein each unit comprises a substrate comprising the benzimidazole; an enteric coating, layered on the substrate; and an outer coating layered on substantially an entirety of the enteric coating which provides protection against humidity and thereby increases chemical stability of the benzimidazole.

According to another aspect of the present invention there is provided a rapidly orally disintegratable composition for a benzimidazole, comprising a tablet having a multiplicity of compressed units, wherein each unit comprises a substrate comprising the benzimidazole; an enteric coating, layered on the substrate; and an outer coating layered on substantially an entirety of the enteric coating. Without wishing to be limited by a single hypothesis, it is believed that the presence of the outer coating provides sufficiently increased stability to the benzimidazole such that it is optionally and preferably not necessary to use a subcoat.

According to another aspect of the present invention there is provided a rapidly orally disintegratable composition for a benzimidazole, comprising a tablet having a multiplicity of compressed units, wherein each unit comprises a substrate comprising the benzimidazole; an enteric coating, layered on the substrate; and an outer coating layered on substantially an entirety of the enteric coating which provides good flowability. Again without wishing to be limited by a single hypothesis, the overcoat may improve the flowability of the excipients used in powdered form, by reducing the surface area of the powders and thus reducing the adhesion of the particles to each other. Flowability is important for homogeneity of the tablet content and uniformity of the tablets' weight.

According to further features in any of the above embodiments of the invention, the outer coat optionally comprises a stress absorber.

According to still another aspect of the present invention there is provided a composition for a benzimidazole, comprising a rapidly orally disintegratable tablet having a multiplicity of compressed units, wherein each unit comprises a substrate comprising the benzimidazole; an enteric coating, which is devoid of a plasticizer, layered on the substrate; and an outer coating comprising a stress absorber layered on substantially an entirety of the enteric coating.

According to an additional aspect of the present invention there is provided a composition for a benzimidazole, comprising a rapidly orally disintegratable tablet having a multiplicity of compressed units, wherein each unit comprises a substrate comprising the benzimidazole; an enteric coating layered on the substrate; and an outer coating comprising a stress absorber layered on substantially an entirety of the enteric coating.

According to yet an additional aspect of the present invention there is provided a method for producing a rapidly orally disintegratable composition for a benzimidazole comprising providing a multiplicity of units, wherein each unit comprises a substrate comprising the benzimidazole, an enteric coating layered on the substrate, and an outer coating layered on substantially an entirety of the enteric coating; forming a mixture of the multiplicity of units with a stress absorber; and compressing the mixture to form a tablet.

According to further features of this embodiment of the method of the present invention, the substrate is optionally produced by dissolving the benzimidazole in an aqueous dispersion and spraying the dispersion onto an inert core. Alternatively, the substrate may be produced without an inert core, by mixing the active ingredient with suitable excipients, followed by a process of compression, granulation, extrusion or spheronization.

According to further features in embodiments of the invention wherein the outer coating comprises a stress absorber, the stress absorber may be, for example, at least one of a polysaccharide or cross-linked polysaccharide, starch, microcrystalline cellulose, ethyl cellulose, a peptide or cross-linked peptide, a protein or cross-linked protein, gelatin or cross-linked gelatin, hydrolyzed gelatin or cross-linked hydrolyzed gelatin, collagen or cross-linked collagen, modified cellulose, polyacrylic acid or cross-linked polyacrylic acid, polyvinyls or crosslinked polyvinyls, or polyacrylat and its copolymers.

The cross-linked polysaccharide is optionally and preferably at least one selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

The modified cellulose is optionally and preferably at least one selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Most preferably, the stress absorber is microcrystalline cellulose.

Optionally and preferably, the stress absorber at least partially coats each of the units.

According to still further features in the described preferred embodiments, the stress absorber is optionally the sole excipient in the outer coating.

Alternatively, the outer coating may comprise an additional excipient.

The additional excipient is optionally and preferably at least one of a binder, a filler, a disintegrant, and an effervescent.

The binder is optionally and preferably at least one of Povidone (PVP: polyvinyl pyrrolidone), low molecular weight HPC (hydroxypropyl cellulose), low molecular weight HPMC (hydroxypropyl methylcellulose), carboxy methyl cellulose, hydroxylethyl cellulose, ethylcellulose, gelatin polyethylene oxide, acacia, dextrin, magnesium aluminum silicate, starch, and polymethacrylates.

The filler is optionally and preferably at least one of sugars such as lactose, glucose, fructose, or sucrose; dicalcium phosphate; sugar alcohols such as sorbitol, manitol, mantitol, lactitol, xylitol, isomalt, erythritol, and hydrogenated starch hydrolysates; corn starch, potato starch, sodium carboxymethycellulose, ethylcellulose and cellulose acetate, or a mixture thereof.

The disintegrant is optionally and preferably at least one of low-substituted carboxymethyl cellulose sodium, cross-linked polyvinyl pyrrolidone, sodium starch glycolate, cross-linked sodium carboxymethyl cellulose, pregelatinized starch, microcrystalline starch, water insoluble starch, calcium carboxymethyl cellulose, and low substituted hydroxypropyl cellulose magnesium aluminum silicate.

The outer coating may optionally further comprise at least one of a sweetener, a flavorant, a colorant, and a lubricant and an alkalizing agent.

According to yet an additional aspect of the present invention there is provided a method for producing a composition for a benzimidazole comprising providing a multiplicity of units, wherein each unit comprises a substrate comprising the benzimidazole, and an enteric coating layered on the substrate; forming a mixture of the multiplicity of units with an adhesive polymer; and shaping the mixture to form a tablet.

Optionally, and preferably, the adhesive polymer is a polymer glue.

According to further features in any of the embodiments of the invention, the benzimidazole is optionally and preferably at least one of omeprazole, lansoprazole and pantoprazole. More preferably, the benzimidazole is lansoprazole.

The benzimidazole may optionally comprise benzimidazole base. Alternatively, the benzimidazole may comprise a benzimidazole salt, such as, for example, the magnesium or sodium salt of omeprazole, or the sodium sesquihydrate of pantoprazole.

According to further features in any of the embodiments of the invention, the substrate optionally and preferably comprises a neutral core and an active coating containing the benzimidazole, the active coating being layered over the neutral core. The neutral core may comprise, for example, at least one of a non-pareil, a bead, a seed, a granule, or a pellet.

The non-pareil is optionally and preferably in the range of from about 80 to about 850 microns. More preferably, the non-pareil is in the range of from about 200 to about 250 microns.

Optionally and preferably, the substrate comprises an aqueous solvent.

According to further features in any of the embodiments of the invention, the enteric coating optionally and preferably comprises at least one enteric material selected from the group consisting of hydroxypropyl methylcellulose acetate succinate (hypromellose acetate succinate), cellulose acetate phthalate, hydroxypropyl methyl cellulose phthalate, polyvinyl acetate phthalate, sodium alginate, alginic acid, poly(methacrylic acid, methyl methacrylate) 1:1 and (Eudragit L100), poly(methacrylic acid, ethyl acrylate) 1:1 (Eudragit L30D-55).

Optionally and preferably, the enteric coating further comprises an organic solvent. More preferably, the enteric solvent comprises acetone. The enteric coating may optionally further comprise at least one excipient, such as, for example, a glidant, lubricant and anti-adherents, including but not limited to talc or titanium dioxide.

According to further features in any of the embodiments of the invention, each of the units optionally and preferably further comprises a sub-coating layered between the substrate and the enteric coating.

The sub-coating optionally and preferably further comprises an aqueous solvent.

Optionally and preferably, one or more of the substrate, and the sub-coating may further comprise an excipient.

The excipient may be at least one of a binder, a surfactant, a filler, a solubilizer, and an alkalinizing agent.

Examples of binders include water-soluble, hydrophilic polymers, such as, for example, Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates, or a mixture thereof.

More preferably, the binder is hydroxypropyl methylcellulose (HPMC).

Examples of surfactants include polysorbate 80 (Tween 80) and sodium lauryl sulfate.

Examples of fillers include, for example, a sugar, such as lactose, glucose, fructose, or sucrose; dicalcium phosphate; sugar alcohols such as sorbitol, manitol, mantitol, lactitol, xylitol, isomalt, erythritol, and hydrogenated starch hydrolysates; corn starch; potato starch; sodium carboxymethycellulose, ethylcellulose and cellulose acetate, or a mixture thereof.

More preferably, the filler is lactose.

Examples of alkalinizing agents include sodium stearate, meglumine, disodium phosphate, and ammonia.

More preferably, the alkalinizing agent comprises sodium stearate or meglumine.

According to another embodiment of the present invention, there is provided a rapidly orally disintegratable tablet having a multiplicity of compressed units for an active ingredient with a bitter or unpleasant taste, comprising a plurality of units, wherein each unit comprises a substrate comprising the active ingredient; an enteric coating layered on the substrate; and an outer coating layered on substantially an entirety of the enteric coating; wherein the outer coating features a taste masking ingredient for masking the taste of the active ingredient. Such a taste masking ingredient may optionally comprise a flavorant or sweetener as described herein.

The term “substrate” refers to substantially any structure which features the benzimidazole derivative, such as lansoprazole.

By “compressed units” it is meant units which have been subjected to sufficient compressional force to form a firm, cohesive tablet.

The phrase “stress absorber” refers to a material which is able to absorb a force applied to the outer coat, thereby preventing the force from being exerted on the enteric coat, and thus protecting the integrity of the enteric layer. This can be achieved by including in the outer coat a polymer having a suitable level of plasticity, or an appropriate particle structure and texture, or both.

When a polymer is exposed to a compressive load which causes the polymer to undergo deformation, the polymer behaves mechanically according to its stress/strain curve which is a mechanical finger print for that specific polymer. Accordingly, the mechanical behavior may be divided into two distinct regions: an elastic region (elastic deformation), and a plastic region (plastic deformation).

If the load is such that stress falls in the elastic region, then according to Hooke's law the strain will be proportional to stress (this proportionality can be expressed by a constant called Young's modulus). In such a case, if the load is removed the polymer can revert back to its original dimensions (a process known as elastic recovery, which is the percent of strain recovered when the load is released). Such a deformation is called elastic deformation. Elastic recovery increases with cross-linking, and may decrease as strain or stress increases.

If the material is stretched too far, Hooke's law ceases to hold and there will be permanent deformation known as plastic deformation. The point at which the stress-strain relationship departs from linear is called the “proportional or elastic limit” (yield point). Once the material has been stressed beyond the proportional limit, a permanent strain is present, even when the stress is reduced to zero. This process is called creep. Elastic materials generally have a larger region of linearity, and therefore a higher yield point. Deformation after the Proportional Limit contains recoverable strain (elastic), and non recoverable strain (plastic). The latter occurs as a result of the creep, and the process is accompanied by release of heat (energy loss).

The deformed stress absorber is energy rich, and this energy is released when the stress absorber is exposed to water. Without wishing to be limited by a single hypothesis, it is believed that the polymer or polymer mixture of the outer coating according to the present invention experiences both types of deformation. The plastic deformation causes the units to bind together as the polymer or polymer mixture of the outer coating on a unit may literally be pushed into that of another unit. However (and without wishing to be limited by a single hypothesis), at other points on the unit, the polymer or polymer mixture of the outer coating may undergo elastic deformation, which may absorb the stress and thereby preventing it from being exerted on the enteric coat, thereby preserving the integrity of the outer coating. The outer coating may optionally provide an additional layer of protection, for example against the entry of moisture through the outer coating and to the enteric coating which may occur during storage for example.

The phrase “enteric coating” refers to a layer which provides protection of the active ingredient against the acid environment of the stomach.

Hereinafter, the term “alkalinizing agent” includes any material which is capable of providing a pH value of at least about 7.0 when present alone in water, preferably at least about 7.5 and more preferably at least about 8.0.

By “rapidly orally disintegratable” is meant that the tablet disintegrates upon oral administration, either in the mouth or upon swallowing, preferably prior to reaching the gastrointestinal tract. Disintegration would generally (optionally and preferably) occur within 60 seconds of administration of the tablet.

As used herein the term “about” refers to ±10%.

### DESCRIPTION OF THE PREFERRED EMBODIMENTS

The present invention is of a multiple unit tablet formulation for a benzimidazole, which is rapidly disintegratable upon oral administration, and can be formulated without the use of a plasticizer.

The principles and operation of the compositions and methods according to the present invention may be better understood with reference to the accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention provides a rapidly orally disintegratable multiple unit tablet composition for a benzimidazole. The tablet comprises a multiplicity of units, consisting of a number of small, individual particles, which are compressed into a cohesive tablet. Compression involves subjecting the multiplicity of units, such as at least two individual units, to a crushing force.

According to a preferred embodiment of the present invention, each unit comprises a substrate comprising a benzimidazole; an enteric coating, which is devoid of a plasticizer, layered on the substrate; and an outer coating layered on the enteric coating, such that the enteric coating of each individual unit is completely covered.

According to an alternative embodiment of the present invention, each unit comprises a substrate comprising a benzimidazole; an enteric coating layered on the substrate; and an outer coating, which serves to protect the integrity of the enteric coating during compression, layered on the substrate, such that the enteric coating of each individual unit is completely covered.

According to an additional alternative embodiment of the present invention, each unit comprises a substrate comprising a substrate comprising a benzimidazole; an enteric coating, layered on the substrate; and an outer coating layered on the enteric coating, such that the enteric coating of each individual unit is completely covered, and which prevents direct contact between the units and thus protects the integrity of the enteric coating.

According to an additional alternative embodiment of the present invention, each unit comprises a substrate comprising a substrate comprising a benzimidazole; an enteric coating, layered on the substrate; and an outer coating layered on the enteric coating, such that the enteric coating of each individual unit is completely covered, and which provides protection against humidity and thereby increases chemical stability of the benzimidazole.

According to an additional alternative embodiment of the present invention, each unit comprises a substrate comprising a substrate comprising a benzimidazole; an enteric coating, layered on the substrate; and an outer coating layered on the enteric coating, such that the enteric coating of each individual unit is completely covered, and which provides good flowability as described above.

Any of the above embodiments may optionally and preferably comprise a stress absorber.

According to a further alternative embodiment of the present invention, each unit comprises a substrate comprising a benzimidazole; an enteric coating, which is devoid of a plasticizer, layered on the substrate; and an outer coating comprising a stress absorber layered on the substrate, such that the enteric coating of each individual unit is completely covered.

According to a further alternative embodiment of the present invention, each unit comprises a substrate comprising a benzimidazole; an enteric coating layered on the substrate; and an outer coating comprising a stress absorber layered on the substrate, such that the enteric coating of each individual unit is completely covered.

The present invention also provides a method for producing a rapidly orally disintegratable composition for a benzimidazole, which method comprises providing a multiplicity of units, each unit comprising a substrate comprising the benzimidazole, on which is layered an enteric coating, such that the enteric coating of each individual unit is completely covered; forming a mixture of the units with a stress absorber; and compressing the mixture to form a tablet.

The present invention also provides a method of producing a rapidly orally disintegratable composition for a benzimidazole, which method comprises providing a multiplicity of units, each unit comprising a substrate comprising the benzimidazole, on which is layered an enteric coating, such that the enteric coating of each individual unit is completely covered; forming a mixture of the units with an adhesive polymer; and shaping the mixture to form a tablet. No compression step is involved in the process according to this embodiment of the present invention. The adhesive polymer may optionally be provided in the outer coating layer.

The composition of the present invention comprises as active ingredient a benzimidazole, such as omeprazole, lansoprazole, or pantoprazole, optionally in the form of a base. Alternatively, the benzimidazole may comprise a single enantiomer of a benzimidazole, or an alkaline benzimidazole salt, such as, for example, the magnesium or sodium salt of omeprazole, or the sodium sesquihydrate of pantoprazole, or one of its single enantiomers. Most preferably, the benzimidazole is lansoprazole, or a salt thereof.

### Substrate

The term “substrate” refers to substantially any structure which features the benzimidazole derivative, such as lansoprazole. For example, this structure could be an active core containing the benzimidazole derivative. This active core could be prepared in a number of different ways which are known in the art. For example, the active core could be formed by compressing the benzimidazole derivative with an alkaline substance. As another example, the active core could be prepared by mixing the benzimidazole derivative with an alkaline substance, spheronizing the mixture and then forming cores through pelletisation. As yet another example, the active core is optionally and preferably prepared by embedding the active ingredient in a poloxamer and compressing the embedded material into tablets. The active core is also optionally formed by granulating the active ingredient with an alkaline substance and compressing the granulation into tablets.

Alternatively and optionally and preferably, the structure could include a neutral core, such as a sugar bead which does not contain the benzimidazole derivative, over which the benzimidazole derivative is coated. The coating includes lansoprazole or other benzimidazole derivative with a suitable adhesive polymer. The neutral core may optionally comprise at least one of a non-pareil, a bead, a seed, a granule, and a pellet. Preferably, the core comprises a non-pareil or a pellet. The pellet optionally and preferably comprises microcrystalline cellulose. More preferably, the non-pareil has a size in the range of from about 80 to about 850 microns, most preferably in the range of from about 200 to about 250 microns. The coating on the substrate of the present invention optionally further comprises an aqueous solvent.

The substrate may optionally comprise at least one excipient, such as a binder, a surfactant, and a filler.

Examples of suitable binders include but are not limited to water-soluble, hydrophilic polymers, such as Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, hydroxyethylcellulose, gelatin, polyethylene oxide, acacia, dextrin, magnesium aluminum silicate, starch, and polymethacrylates. Optionally and preferably, the binder comprises hydroxypropyl methylcellulose or povidone.

Examples of suitable fillers include but are not limited to lactose, glucose, fructose, sucrose, dicalcium phosphate, sugar alcohols also known as “sugar polyol” such as sorbitol, manitol, mantitol, lactitol, xylitol, isomalt, erythritol, and hydrogenated starch hydrolysates (a blend of several sugar alcohols), corn starch, potato starch, sodium carboxymethycellulose, ethylcellulose and cellulose acetate, or a mixture thereof. Optionally and preferably, the filler comprises lactose.

Examples of suitable surfactants include but are not limited to polysorbate 80 (for example Tween 80, or sodium lauryl sulfate.

The substrate may optionally comprise an alkalinizing agent, such as, for example, an inorganic basic salt, such as basic inorganic salts of sodium; magnesium or calcium, (such as sodium hydrogen carbonate, sodium stearate, disodium phosphate), meglumine, or ammonia. Examples of such basic inorganic salts of magnesium include, but are not limited to, heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite [Mg₆Al₂(OH)₁₆.CO₃.4H₂O] and aluminum magnesium hydroxide [2.5MgO.Al₂O₃.xH₂O]. Examples of such basic inorganic salts of calcium include, but are not limited to, precipitated calcium carbonate and calcium hydroxide.

Optionally and preferably, the alkalinizing agent comprises sodium stearate or meglumine.

### Subcoating

Optionally and preferably, a formulation according to the present invention features a subcoating layer between the substrate and the enteric coating layer. The sub-coating layer is provided in order to prevent interaction between the enteric coating layer and the substrate containing the benzimidazole, particularly in embodiments wherein the substrate includes an alkalinizing agent. The benzimidazole-containing alkaline reacting substrate is preferably separated from the enteric coating polymer(s) containing free carboxyl groups, which otherwise causes degradation/discoloration of the benzimidazole during the coating process or during storage.

The subcoating layer may optionally comprise at least one excipient, such as a binder, a surfactant, and a filler.

Examples of suitable binders include but are no limited to water-soluble, hydrophilic polymers, such as Povidone (PVP: polyvinyl pyrrolidone), low molecular weight HPC (hydroxypropyl cellulose) low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally and preferably, the binder comprises HPMC.

Examples of suitable fillers include but are not limited to lactose, glucose sucrose, sorbitol, dicalcium phosphate, manitol, mantitol, corn starch, potato starch, sodium carboxymethycellulose, ethylcellulose and cellulose acetate. Optionally and preferably, the filler comprises lactose.

Examples of suitable surfactants include but are not limited to polysorbate 80 (for example Tween 80 or sodium lauryl sulfate). Optionally and preferably, the surfactant comprises polysorbate 80.

Alternatively, the subcoating layer may comprise Opadry II HP, based on polyvinyl alcohol.

The subcoating layer may optionally comprise an alkalinizing agent, such as, for example, sodium stearate, meglumine, disodium phosphate, or ammonia. Optionally and preferably, the alkalinizing agent is either sodium stearate or meglumine.

Examples of such basic inorganic salts of magnesium include, but are not limited to, heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite [Mg₆Al₂(OH)₁₆.CO₃.4H₂O] and aluminum magnesium hydroxide [2.5MgO.Al₂O₃.xH₂O]. Examples of such basic inorganic salts of calcium include, but are not limited to, precipitated calcium carbonate and calcium hydroxide.

### Enteric Coating

The formulation according to the present invention optionally and preferably features an enteric coating, which comprises at least one enteric coating material. The enteric coating material is preferably a pH dependent polymer, more preferably a polymer selected from the group consisting of hydroxypropyl methylcellulose acetate succinate (also known as hypromellose acetate succinate), cellulose acetate phthalate, hydroxypropyl methyl cellulose phthalate, polyvinyl acetate phthalate, poly(methacrylic acid, methyl methacrylate) 1:1 and poly(methacrylic acid, ethyl acrylate) 1:1, alginic acid, and sodium alginate. A suitable enteric coating can be made from Eudragit™ polymers series (available from Rohm Pharma) which are polymeric lacquer substances based on acrylates and/or methacrylates. Suitable polymers which are slightly permeable to water, and exhibit a pH-dependent permeability include, but are not limited to, Eudragit™ S (poly(methacrylic acid, methyl methacrylate) 1:2); Eudragit L100™ (poly(methacrylic acid, methyl methacrylate) 1:1); Eudragit L30D™, (poly(methacrylic acid, ethyl acrylate) 1:1); and (Eudragit L100-55) (poly(methacrylic acid, ethyl acrylate) 1:1). Eudragit™ L is an anionic polymer synthesized from methacrylic acid and methacrylic acid methyl ester. It is insoluble in acids and pure water. It becomes soluble in neutral to weakly alkaline conditions. The permeability of Eudragit™ L is pH dependent. Above pH 5.0, the polymer becomes increasingly permeable. Mixtures of such polymers may also optionally be used.

Optionally and preferably, the enteric polymer comprises HPMC acetate succinate.

The enteric coating optionally further comprises an organic solvent, such as acetone. The enteric coating optionally further comprises an excipient, such as, for example, a glidant, such as talc or titanium dioxide.

### Outer Coating

The outer coating of the composition according to the present invention substantially entirely covers the enteric coating of each individual unit. This prevents direct contact between individual units in one hand and on the other hand absorbs the stress resulting from the compression force, all of which assists in protecting the integrity of the enteric coating of the units, increases flowability, prevents segregation, and provides excellent protection against humidity, thereby increasing the chemical stability of the benzimidazole. Additionally, the outer coating of the present invention enables fast disintegration of the composition, while still permitting direct compression of the units of the composition into a tablet without requiring the addition of further tablet excipients.

The outer coating of the composition according to the present invention optionally and preferably features a stress absorber. The stress absorber may be added to the outer coating prior to layering of the outer coating over the enteric coating layer.

According to some embodiments of the present invention, the stress absorber may optionally and preferably at least partially coat the units of the composition.

As described in greater detail above, a stress absorber according to the teachings of the present invention is a material having a high degree of plasticity, such that the material can easily undergo deformation under stress, releasing the stress as heat, thereby enabling stress relaxation to occur during compression of the units of the composition to form a tablet. This prevents cracking of the enteric coating during the compression process.

The stress absorber according to any of the embodiments of the present invention may optionally comprise one of polysaccharides or cross-linked polysaccharides, starch, microcrystalline cellulose, ethyl cellulose, peptides or cross-linked peptides, protein or cross-linked proteins, gelatin or cross-linked gelatin, hydrolyzed gelatin or cross-linked hydrolyzed gelatin, collagen or cross-linked collagen, modified cellulose, polyacrylic acid or cross-linked polyacrylic acid, polyvinyls (such as polyvinylalcohol, polyvinyl acetate and polyvinyl pyrrolidone and their copolymers) or cross-linked polyvinyls, polyacrylat and its copolymers (such as Eudragit RL, Eudragit RS, Eudragit E, Eudragit L) or cross-linked polyacrylats. The cross-linked polysaccharide can be selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof. The modified cellulose may be selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Optionally and preferably, the stress absorber comprises microcrystalline cellulose.

The inclusion of a stress absorber in the outer coating prevents compression pressure from being exerted on the enteric coating. This abrogates the need for a plasticizer in the enteric coating, thereby avoiding the disadvantages associated with plasticizers, as discussed in greater detail in the Background section above. Hence, preferably, the enteric coating of a unit of the present invention is devoid of a plasticizer.

According to some of the embodiments wherein the outer coating comprises a stress absorber, the stress absorber may optionally be provided as the sole excipient in the outer coating.

Alternatively, the outer coating may comprise at least one further excipient, in addition to the stress absorber. Non-limiting examples of suitable excipients are described below. For example, the outer coating may optionally include a binder selected from the group including but not limited to a water-soluble hydrophilic polymer, such as Povidone (PVP: polyvinyl pyrrolidone), low molecular weight hydroxypropyl cellulose (HPC), low molecular weight hydroxypropyl methylcellulose (HPMC), low molecular weight carboxy methyl cellulose, hydroxyethylcellulose, gelatin, polyethylene oxide, acacia, dextrin, magnesium aluminum silicate, starch, and polymethacrylates.

Optionally and preferably, the concentration of the binder in the outer coating is in the range of from about 2 to about 15% w/w of the total dry outer coating, and the concentration of stress absorber in the outer coating is in the range of from about 10 to about 50% w/w of the total dry outer coating.

The outer coating may optionally and preferably feature one or more fillers, optionally selected from the group including but not limited to sugars, such as lactose, glucose, fructose, sucrose, dicalcium phosphate, sugar alcohols such as sorbitol, manitol, mantitol, lactitol, xylitol, isomalt, erythritol, and hydrogenated starch hydrolysates, corn starch, potato starch, sodium carboxymethycellulose, ethylcellulose and cellulose acetate, Pharmaburst® (a disintegrant based on mannitol) or a mixture thereof.

Optionally and preferably, the concentration of the filler in the outer coating is in the range of from about 30 to about 70% w/w of the total dry outer coating.

The outer coating may also optionally and preferably feature one or more disintegrants, optionally selected from the group including but not limited to low-substituted carboxymethyl cellulose sodium, crospovidone (cross-linked polyvinyl pyrrolidone), sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, low substituted hydroxypropyl cellulose, and magnesium or aluminum silicate. The composition of the present invention may optionally comprise at least two types of pellets, having the same substrate, subcoating layer and enteric coating, but differing in that a first of the two types of pellets includes a disintegrant in the outer coating, while a second of the two types of pellets is devoid of a disintegrant in the outer coating.

The mechanism of disintegration is based on swelling, wicking, and deformation of the disintegrants. Some commercial superdisintegrants for use in the present invention include but are not limited to Ac-Di-Sol, Primojel, Explotab, and Crospovidone.

The disintegrant preferably constitutes from about 2 to about 10% of the total solid weight of the outermost layer.

The outer coating preferably features an effervescent. An effervescent material is optionally and preferably included in order to increase dissolution rate of the other excipients. The term “effervescent” includes compounds which evolve gas. The preferred effervescent agents evolve gas by means of a chemical reaction, such as between an acid and a base, which takes place upon exposure of the effervescent to water and other fluids. Such water-activated materials must be kept in a generally anhydrous state and with little or no absorbed moisture or in a stable hydrated form, since exposure to water will prematurely disintegrate the tablet.

The acid may be any which is safe for human consumption and may generally include but is not limited to food acids, acid and hydrite antacids including but not limited to, for example, citric, tartaric, malic, fumaric, adipic, and succinic.

The base may comprise a carbonate source. Carbonate sources include dry solid carbonate and bicarbonate salt such as, preferably, sodium bicarbonate, sodium carbonate, potassium bicarbonate and potassium carbonate, magnesium carbonate and the like. Carbonate sources such as sodium bicarbonate are preferable, in that they may also serve as an alkalinizing agent. Optionally and preferably, the concentration of the effervescent in the outer coating is in the range of from about 5 to about 15% w/w of the total dry outer coating.

The composition of the present invention may optionally comprise at least two types of pellets, having the same substrate, subcoating layer and enteric coating, but differing in the composition of the outer coating, such that a first of the two portions includes an acid and a second of the two portions includes a base. For example, the outer coating of a first portion of pellets may comprise sodium carbonate and the outer coating of a second portion of pellets may comprise tartaric acid.

The outer coating may also optionally further comprise an additional excipient, such as one which increases palatability. Examples of additional excipients include a sweetener (such as acesulfame potassium), a flavorant (such as orange or mint flavor, or a combination thereof), a colorant, and a lubricant to ease in swallowing (such as polyethylene glycol). Flavorants and sweeteners are particularly useful when the active ingredient has a bitter taste, the masking of which would increase patient compliance.

The outer coating may optionally and preferably feature one or more alkalizing agents, optionally selected from the group including but not limited to sodium stearate, meglumine, disodium phosphate, and ammonia

The outer coating of the present invention enables fast disintegration of the composition; allows direct compression of the units of the composition into a tablet without requiring the addition of further tablet excipients; and improves the stability of the pellets, which undergo discoloration in the absence of an outer coating layer. Fast or rapidly disintegrating means disintegration that occurs within not more than about 15 minutes.

### Preparation of Formulations According to the Present Invention

The preparation of the compositions of the present invention is described first with reference to the following general description and then with reference to the following non-limiting examples of the preparation and application of the compositions of the present invention.

As noted previously, the composition of the present invention includes a substrate which features a benzimidazole. The substrate may be an active core containing the benzimidazole. This active core could be prepared in a number of different ways which are known in the art. For example, the active core could be formed by compressing benzimidazole with at least one excipient. As another example, the active core could be prepared by mixing the benzimidazole with the additional ingredient(s), spheronizing the mixture and then forming cores through pelletisation. The active core is also optionally formed by granulating the active ingredient with the additional ingredient(s) and compressing the granulation into tablets. The active core is also optionally formed by preparing pellets, and then compressing the pellets into a tablet.

The substrate is preferably prepared by dissolving the benzimidazole in an aqueous solvent, optionally also including at least one of a filler, a surfactant, a binder, a solubilizer and an alkalinizing agent. This solution is then applied to an inert core, such as, for example, a non-pareil, a bead, a seed, a granule or a pellet.

The active layer may optionally be applied to the inert core by a dry coating process (dry powder layering), as discussed in detail below. Dry powder layering is particularly advantageous for application of the lansoprazole-containing active layer, since many excipients have been found to be incompatible with lansoprazole. This incompatibility is generally more apparant in a liquid medium such as a coating solution or suspension. In contrast, dry powder coating technology may provide a more stable manufacturing method for acid-labile drugs such as lansoprazole. For dry coating, lansoprazole can be applied to either sugar or microcrystalline spheres using an appropriate dry coating machine. The dusting powder, including the active material can be applied individually while spraying a binder solution. This dusting powder may further contain additional excipients, such as stabilizers, buffering agents, fillers, glidants, lubricants, surface active agents, solubilizers, dispersing agents, emulsifying agents, wetting agents, suspending agents, disintegrants, binders, and combinations thereof. Additionally, a second dusting powder can be applied to the pellets to enhance drug protection. This second dusting powder may contain fillers, disintegrants and binders.

Alternatively, the substrate may optionally be prepared without an inert core, by compression or wet granulation of these ingredients, or extrusion and spheronisation, or through any other suitable preparation method thereof.

Further alternatively, the substrate may be prepared by spheronization, such that the active material is encapsulated within a microsphere.

The subcoating layer is then coated over the substrate. Preferably, the subcoating layer is prepared by adding an organic basic salt, more preferably sodium stearate, as the alkaline agent, to an aqueous solution. Alternatively, the alkaline agent could be an inorganic basic salt as described below. The solution may also optionally include other ingredients, such as one or more surfactants, and/or one or more fillers, and/or one or more cellulosic polymers.

The subcoating layer can be applied to the substrate by conventional coating techniques such as, for instance, pan coating, fluidized bed coating, fluidized bed bottom sprayed coating or a Turbo Jet-Technology for the production of large amounts. Coating may be performed using a Fluidized Bed Processor (such as that of Glatt Gmbh), Unilab Fluidized Bed (Huttilin), or Ventilus Fluid Bed (Innojet). A fluidized bed is a bed of solid particles which are suspended in a stream of air or gas passing upward through the particles, in which the coating material is aerosolized. As the air travels through the particle bed, the particles are mixed in the stream of gas or air with the coating material, and so are coated but are also dried. Alternatively a turbo coating system may optionally be used.

Further alternatively, dry powder layering may be used for the subcoating layer. This process provides a number of advantages over conventional, liquid-based coating techniques. Dry powder layering has the advantage of enabling use of specific excipients while keeping their original properties. For example, it is well known that use of super-disintegrants or burst controlling agents in fast dissolving formulations may improve the disintegration rate. Using such materials in an aqueous-based coating formulation may, however, negatively alert their original properties, eventually causing a longer disintegration time. This problem may be totally overcome by use of a dry powder coating process.

A dry coating process also results in much lower energy requirements, more efficient utilization of coating materials, greater environmental friendliness, and lower operating costs, as compared to liquid-based methods. Pans used in aqueous coatings systems may be used for dry powder coatings processes, with only minor modifications. Since in dry powder coating, little or no solvent or water is used, it can be considered a more economical process than liquid coating processes, since vaporizing the liquid requires considerable energy consumption. Small dosage forms such as pellets and particles are currently coated in fluidized beds, which requires even larger amounts of hot air, and which may strengthen the concern about energy consumption when using a liquid-based or wet coating process. Use of organic solvents further results in environmental pollution, high solvent recycling costs, and danger of explosion during operation. Likewise, when using a wet coating process, air cleaning may also be a huge burden on the process, as the hot air has to be cleaned at both intake and outlet stages.

Dry powder coating processes may be carried out using many known systems, such as, for example, CF-Granulator (Freund Industrial, Tokyo, Japan), Granurex (Vector Corporation, Marion, Iowa, USA), GS HP/25 equipment (GS Coating System, Italy), Centrifugal Fluid Bed Granulator (Glatt, Germany) and other appropriate systems. A solution is then prepared with the enteric coating material. The solution preferably includes a solvent or a mixture thereof, including but not limited to, an aqueous solvent such as water, or an organic solvent such as isopropyl alcohol or other alcohols such as ethanol, or acetone. Mixtures of aqueous and organic solvents preferably include at least one polar organic solvent such as isopropyl alcohol for example. The solution may also optionally and preferably include a plasticizer, and/or a binder, and/or a surfactant.

This enteric coating solution is then layered over the previously coated (with the subcoating material) substrate to form the composition of the present invention. Any of the coating techniques described above for application of the subcoating layer may be used for layering of the enteric coating solution, including dry powder coating.

The outer coating layer is then layered over the enteric coating layer, again using any of the coating methods described above. Optionally, the enteric coated pellets may first be divided into at least two portions, and different coating layers applied to each of the two portions. For example, a first portion may be coated with a layer comprising an effervescent, while a second portion is coated with a layer devoid of an effervescent. The coating layers may further differ in other excipients, such as, for example, in flavorants.

If dry powder coating techniques are to be used for the outer coating layer, the enteric coated pellets may be place in a coating system, and the dry powder applied while simultaneously spraying with a binder solution. A wide range of concentrations of binder solution may be used, with either aqueous or organic solvents. The binder solution may be continuously sprayed onto the moving pellets using a peristaltic pump. Addition of the powder may begin either at the same time, or shortly after, spraying of the binder solution begins. At the end of the process, the resultant over-coated pellets may be dried for an additional period of time prior to discharging, in order to allow any residues of the solvent to be vaporized as much as possible.

The rate, amount, homogeneity, inter- and intra-uniformity, efficiency, quality, and yield of the coating may be controlled by parameters such as batch size, rotor speed, binder spray rate, powder addition rate, inlet and outlet air temperature, bed temperature, atomization air pressure, air flap and air flow.

According to another optional embodiment of the present invention, the coating suspension was prepared as follows. First hydroxypropyl cellulose (HPC) (8 g) was dissolved in purified water (600 g) to obtain a clear solution. Then PEG-2000 (6.7 g), sodium bicarbonate (14.1 g) and acesulfame potassium (1.3 g) were added to the HPC solution and mixed to complete dissolution. Sorbitol (91.7 g), microcrystalline cellulose (49 g), crospovidone (9 g) and starch 1500 (5.5 g) were then added to the resulting clear solution and mixed with a Heidolph mixer to obtain a homogeneous suspension which was stirred throughout the coating process. The coating can be applied to the pellets by conventional coating techniques such as, for instance, pan coating, fluidized bed coating, fluidized bed bottom sprayed coating or a Turbo Jet-Technology for the production of large amounts. Coating may be performed using a Fluidized Bed Processor (such as that of Glatt Gmbh). A fluidized bed is a bed of solid particles which are suspended in a stream of air or gas passing upward through the particles, in which the coating material is aerosolized. As the air travels through the particle bed, the particles are mixed in the stream of gas or air with the coating material, and so are coated but are also dried. Alternatively a turbo coating system may optionally be used.

The coating solution or suspension may be based on dispersions in water and/or suitable organic solvents or by using latex suspensions of the polymers. Examples of enteric coating polymers are as given above.

The coated units are then compressed into a tablet, using any tabletting device known in the art. Compression is preferably performed at room temperature. “Room temperature” used herein refers to a temperature in a room at which compression is performed in manufacturing of the tablet, and the temperature is usually in the range of from about 20° C. to about 23° C.

Compositions of the present invention may, if desired, be presented in a pack or dispenser device, such as an FDA approved kit, which may contain one or more dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non limiting fashion.

### Example 1

### A. Substrate

(i) Inert core: sugar beads (200-250 microns)
(ii) Active layer:

| | | Amount (% w/w of the total |
|---|---|---|
| Ingredient | Function | dry active layer weight) |
| lansoprazole | active agent | 34.7 |
| HPMC | binder | 29.0 |
| polysorbate 80 | surfactant | 5.8 |
| lactose | filler | 29.0 |
| sodium stearate | alkalinizing agent | 1.5 |
| water | solvent | Not present in final product |

HPMC is dissolved in purified water, until completely dissolved to form a first solution. A second solution is prepared by adding polysorbate 80, sodium stearate and lactose to purified water, until completely dissolved, after which lansoprazole is added to the solution. The first solution is then added to the second solution to form an active coating solution.

A fluidized bed coating device (Glatt, Germany) is loaded with the microcrystalline cellulose pellets. The active coating solution is sprayed on the pellets, using standard coating techniques, to form an active substrate.

Typical process parameters are: inlet temperature 50-55° C.; automizing air pressure 0.8-1.3 bar; microclimate pressure 0.8-1.0 bar; product temperature 28-30° C.; outlet air pressure 28-30° C.

### B. Subcoating Layer

| | | Amount (% w/w of the total |
|---|---|---|
| Ingredient | Function | dry subcoating layer weight) |
| HPMC | binder | 44.6 |
| polysorbate 80 | surfactant | 8.8 |
| lactose | filler | 44.6 |
| sodium stearate | alkalinizing agent | 2 |
| water | solvent | Not present in final product |

The subcoating layer is applied to the active substrate, using standard spraying techniques, such as those described above.

### C. Enteric Coating

| | | Amount (% w/w of the total |
|---|---|---|
| Ingredient | Function | dry enteric coating weight) |
| HPMC acetate succinate | enteric material | 100 |
| acetone | solvent | Not present in final product |
| water | solvent | Not present in final product |

The enteric coating is applied over the subcoating layer, using standard spraying techniques, such as described in section A above.

### D. Outer Coating

| | | Outer coating |
|---|---|---|
| | | Amount (% w/w of the total |
| Ingredient | Function | dry outer coating weight) |
| microcrystalline cellulose | stress absorber | 26.5 |
| sorbitol | filler | 49.5 |
| croscarmellose | disintegrant | 4.9 |
| Starch 1500 | disintegrant | 2.9 |
| HPC | binder | 4.3 |
| sodium bicarbonate | effervescent | 7.6 |
| acesulfame potassium | sweetener | 0.7 |
| PEG 2000 | lubricant | 3.6 |

The coating process was performed in a Fluidized bed Wurster coater (Uni-Glatt CN:6599) using one spray nozzle. The coating process was carried out under the following conditions: air inlet temperature 38-44° C., air outlet temperature 28-36° C., air pressure 2.5-2.6 bar and spray rate 6-12 ml/min.

The coated beads were compressed to 8 mm diameter tablets using a single punch tabletting machine (WICK). The hardness ranged between 12 kilo Newton (kN) and 20 kN.

For hardness of 12 kN, disintegration time was 1-3 minutes in buffer of pH 6.8 in a disintegration device, and 3-40 seconds in the mouth. For hardness of 18-20 kN, disintegration time was 3-5 minutes in buffer, and 1-1.5 minutes in the mouth.

### Example 2

The active layer, subcoating layer, and enteric layer were prepared and applied as for Example 1. The outer coating layer was prepared as follows:

| | | Formulation B |
|---|---|---|
| | | Amount (% w/w of the total |
| Ingredient | Function | dry outer coating weight) |
| microcrystalline cellulose | stress absorber | 28.5 |
| sorbitol | filler | 54.1 |
| HPC | binder | 4.6 |
| sodium bicarbonate | effervescent | 8.2 |
| acesulfame potassium | sweetener | 0.7 |
| PEG 2000 | lubricant | 3.9 |

### Example 3

The active layer, subcoating layer, and enteric layer were prepared and applied as for Example 1. The enteric coated pellets were then divided into two portions. A first portion was coated with outer coating A, and a second portion was coated with outer coating B. As for Example A, except that outer coatings A and B are prepared as follows:

| | | Outer coating | Outer coating |
|---|---|---|---|
| | | A Amount (% | B Amount (% |
| | | w/w of the | w/w of the total |
| | | total dry outer | dry outer |
| Ingredient | Function | coating weight) | coating weight) |
| microcrystalline | stress absorber | 20.5 | 20.7 |
| cellulose | | | |
| Pharmaburst | filler | 40.8 | 41.4 |
| povidone | binder | 3.3 | 3.3 |
| sodium bicarbonate | effervescent | 30.0 | — |
| acesulfame potassium | sweetener | 2.7 | 2.0 |
| PEG 2000 | lubricant | 2.7 | 2.8 |
| Tartaric acid | effervescent | — | 27.0 |
| Orange flavor | Flavoring agent | — | 2.8 |
| water | solvent | Not present | Not present |
| | | in final | in final |
| | | product | product |

After applying the outer coating layers, the two types of coated pellets were mixed in a ratio of 1:1. The pellets were compressed in a tableting machine (Korsch XL-100) to 10.3 mm tablets.

The resulting tablets had the following characteristics:

| Parameter | | |
|---|---|---|
| Tablet weight | 350 | mg |
| Thickness | 3.75 | mm |
| Friability | 0.9% | |
| Disintegration | 2.05 | min |

### Example 4

As for Example 3, except that coating was performed using Huttlin's Unilab fluidized bed equipment. Typical process parameters were: inlet temperature 50-55° C.; atomizing air pressure 1.0-1.8 bar; microclimate pressure 0.2-0.4 bar; product temperature 30-35° C.

### Example 5

As for Example 3, except that coating was performed using Innojet's Ventilus Fluid bed. The process included atomizing air pressure of 0.8-1.4 and support pressure of 0.2-0.5.

### Example 6

Tablets were prepared with a superdisintegrant outer coating layer as follows:

### A. Substrate

(i) Inert core: microcrystalline cellulose pellets (average size 250 microns)
(ii) Active layer:

| | | Amount (% w/w of the total |
|---|---|---|
| Ingredient | Function | dry active layer weight) |
| lansoprazole | active agent | 32.1% |
| HPMC | binder | 24.3% |
| lactose | filler | 24.3% |
| Polysorbate 80 | surfactant | 9.3% |
| meglumine | alkalinizing agent | 10% |
| water | solvent | Not present in final product |
| Total | | 100% |

The active layer is applied to the inert core as described above for Example 2.

### B. Subcoating Layer

| | | Amount (% w/w of the total |
|---|---|---|
| | | dry subcoating layer |
| Ingredient | Function | weight) |
| HPMC | binder | 44.6% |
| lactose | filler | 44.6% |
| polysorbate 80 | surfactant | 8.8% |
| meglumine | alkalinizing agent | 2% |
| water | solvent | Not present in final product |
| Total | | 100% |

The subcoating layer is applied to the active substrate, using standard spraying techniques, such as described in section A above.

### C. Enteric Coating

| | | Amount (% w/w of the total |
|---|---|---|
| Ingredient | Function | dry enteric coating weight) |
| HPMC acetate succinate | enteric material | 100% |
| acetone | solvent | Not present in final product |
| water | solvent | Not present in final product |
| Total | | 100% |

The enteric coating is applied over the subcoating layer, using standard spraying techniques, such as described in section A above.

### D. Outer Coating Layer

| | | Amount (% w/w of the total |
|---|---|---|
| Ingredient | Function | dry enteric coating weight) |
| povidone | binder | 5.9% |
| polyethylene glycol | lubricant | 3.7% |
| croscarmellose sodium | disintergrant | 5.9% |
| mannitol | filler | 42% |
| sorbitol | filler | 13.8% |
| microcrystalline cellulose | stress absorber | 26.1% |
| acesulfame potassium | sweetener | 2.6% |
| water | solvent | Not present in final product |
| | | 100% |

The enteric coated pellets were compressed in a tableting machine (Korsch XL-100) to 10.3 mm or 14.4 mm tablets.

The resulting tablets had the following characteristics:

| Parameter | | |
|---|---|---|
| Tablet weight | 400 | mg |
| Thickness | 4.45 | mm |
| Friability | 5% | |
| Disintegration | 1.45 | min |

Dissolution of the pellets showed resistance in gastric fluids after 1 hour in HCl 0.1N.

Dissolution of the compressed tablets showed release of over 75% within 30 min from buffer change.

### Example 7

The active layer, subcoating layer, and enteric layer were prepared and applied as for Example 6. The outer coating layer was prepared as follows:

| | | Outer coating | Outer coating |
|---|---|---|---|
| | | A Amount (% | B Amount (% |
| | | w/w of the | w/w of the |
| | | total dry outer | total dry outer |
| Ingredient | Function | coating weight) | coating weight) |
| microcrystalline cellulose | stress | 19.5 | 19.5 |
| | absorber | | |
| mannitol | filler | 30.8 | 30.8 |
| sorbitol | filler | 10.3 | 10.3 |
| povidone | binder | 3.3 | 4.4 |
| sodium bicarbonate | effervescent | 30.0 | — |
| acesulfame potassium | sweetener | 2.1 | 1.0 |
| PEG 2000 | lubricant | 2.7 | 2.7 |
| tartaric acid | effervescent | — | 26.9 |
| orange flavor | flavoring | — | 2.2 |
| | agent | | |
| mint flavor | flavoring | — | 2.2 |
| | agent | | |
| water | solvent | Not present in | Not present in |
| | | final product | final product |

The enteric coated pellets were then divided into two portions. A first portion was coated with outer coating A, and a second portion was coated with outer coating B.

After applying the outer coating layers, the two types of coated pellets were mixed in a ratio of 1:1. The pellets were compressed to tablets of diameter 10.3 mm.

The resulting tablets had the following characteristics:

| Parameter | | |
|---|---|---|
| Tablet weight | 362 | mg |
| Thickness | 3.9 | mm |
| Friability | 0.8% | |
| Disintegration | 1.30 | min |

The compressed tablets showed a release of over 75% of the active material within 30 min from the buffer change.

### Example 8

### Bioavailability Study

A randomized, pharmacokinetic pilot study is undertaken to evaluate the bioavailability of test formulations of lansoprazole. For the study, lansoprazole tablets are prepared according to any suitable example above. A clinical study studies the issue of bioavailability. This study compares the efficacy and pharmacokinetic parameters of a tablet according to the present invention, with a MUPS reference product which contains a regular dosage of lansoprazole. It is believed that tablets prepared according to the present invention will show bioequivalence to a commercially available lansoprazole MUPS tablet product.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A composition for a benzimidazole, comprising a rapidly orally or extra-orally disintegratable tablet comprising a multiplicity of compressed units, wherein each of said units comprises:
(i) a substrate comprising the benzimidazole, wherein said substrate comprises a neutral core and an active coating containing said benzimidazole and wherein said active coating being layered over said neutral core;
(ii) an enteric coating layered on said substrate; and
(iii) at least one type of outer coating layered on substantially an entirety of said enteric coating, wherein said enteric coating is devoid of a plasticizer and said outer coating comprising a stress absorber layered on substantially an entirety of said enteric coating;

2. The composition of claim 1, wherein said stress absorber is microcrystalline cellulose.

3. The composition of claim 1, wherein said stress absorber is the sole excipient in said outer coating.

4. The composition of claim 1, wherein said tablet is disintegratable in a medium selected from the group consisting of aqueous solution, water and saliva.

13. The composition of claim 1, wherein said stress absorber is selected from the group consisting of polysaccharides or cross-linked polysaccharides, starch, microcrystalline cellulose, ethyl cellulose, peptides or cross-linked peptides, protein or cross-linked proteins, gelatin or cross-linked gelatin, hydrolyzed gelatin or cross-linked hydrolyzed gelatin, collagen or cross-linked collagen, modified cellulose, polyacrylic acid or cross-linked polyacrylic acid, polyvinyls or cross linked polyvinyls, polyacrylat and its copolymers, and mixtures thereof.

5. The composition of claim 2, wherein said stress absorber comprises microcrystalline cellulose.

6. The composition of claim 1, further comprising an excipient, wherein said excipient comprises at least one of a binder, a filler, a disintegrant, and an effervescent.

7. The composition of claim 1, wherein said multiplicity of compressed units comprises at least two types of pellets, a first type of said pellets and a second type of said pellets, wherein said outer coating of said first type of said pellets comprises an acid and said outer coating of said second type of said pellets comprises a base, wherein said acid and said base comprise said effervescent.

8. The composition of claim 1, wherein said benzimidazole is selected from the group consisting of omeprazole, lansoprazole and pantoprazole.

9. The composition of claim 1, wherein said neutral core comprises a non-pareil, a bead, a seed, a granule, or a pellet.

10. The composition of claim 9, wherein said non-pareil has a size in the range of from about 80 to about 850 microns.

11. The composition of claim 10, wherein said non-pareil has a size in the range of from about 200 to about 250 microns.

12. The composition of claim 1, wherein said substrate comprises an aqueous solvent.

13. The composition of claim 1, wherein said enteric coating comprises at least one enteric material selected from the group consisting of hydroxypropyl methylcellulose acetate succinate (hypromellose acetate succinate), cellulose acetate phthalate, hydroxypropyl methyl cellulose phthalate, polyvinyl acetate phthalate, alginic acid, and sodium alginate, Eudragit S™; Eudragit L 100™; Eudragit L30D™; Eudragit LIOO-55 and Eudragit™ L or mixtures thereof.

14. The composition of claim 13, wherein said enteric coating further comprises an organic solvent.

15. The composition of claim 14, wherein said organic solvent comprises acetone.

16. The composition of claim 1, wherein each of said units further comprises a sub-coating layered between said substrate and said enteric coating.

17. The composition of claim 16, wherein at least one of said substrate, and said sub-coating further comprises an excipient selected from the group consisting of a binder, a surfactant, a filler, a solubilizer, and an alkalinizing agent.

18. The composition of claim 1, for a benzimidazole, wherein said tablet disintegrates rapidly upon contact with moisture.

19. The composition of claim 18, with the proviso that said moisture is not located within the stomach, small intestine or colon.

20. The composition of claim 1, wherein said tablet is suitable for oral administration and is in a substantially disintegrated form before entering the stomach.

21. The composition of claim 1, wherein at least a portion of said multiplicity of compressed units have separated upon entering the gastro-intestinal tract.

22. The composition of claim 6, wherein said binder is selected from a group including water-soluble, hydrophilic polymers, Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates, or any mixture thereof.

23. The composition of claim 22, wherein said binder is hydroxypropyl methylcellulose (HPMC).
